(19) United States
(12) Patent Application Publication
(10) Pub. No.: US 2012/0034300 A1
(43) Pub. Date: Feb. 9, 2012

(54) STABILIZED ZOLPIDEM PHARMACEUTICAL COMPOSITIONS

(76) Inventors: Nikhilesh N. SINGH, Mill Valley, CA (US); Sathasivan Indiran Pather, El Sobrante, CA (US); Pauly Kavalakatt, Fremont, CA (US); Dennis W. Adair, Fairfield, CA (US)

(21) Appl. No.: 13/078,731

(22) Filed: Apr. 1, 2011

Related U.S. Application Data
(63) Continuation of application No. 12/795,107, filed on Jun. 7, 2010, now abandoned.
Application No. 12/795,107 is a continuation of application No. 11/948,259, filed on Nov. 30, 2007, now abandoned.
(60) Provisional application No. 60/868,029, filed on Nov. 30, 2006.

(51) Int. Cl. A61K 9/20; A61P 25/20; A61K 9/14; A61K 31/437; A61K 9/00

(52) U.S. Cl. 424/464; 514/300; 424/400; 424/490

(57) ABSTRACT

Methods of inducing antinociception in a human are described. The method includes the step of administering an effective dose of a polypeptide comprising L-neo-tryptophan to the human extracranially. The polypeptide containing L-neo-tryptophan could be, but is not limited to, NT64L, NT65L, NT66L, NT67L, NT69L, NT69L′, NT71, NT72, NT73, NT74, NT75, NT76, or NT77.

### CROSS-REFERENCES TO RELATED APPLICATIONS

This is a continuation of U.S. application Ser. No. 12/795,107, filed on Jun. 7, 2010, which is a continuation of U.S. application Ser. No. 11/948,259, filed Nov. 30, 2007, which claims the benefit of U.S. Provisional Application Ser. No. 60/868,029, filed Nov. 30, 2006, entitled “Stabilized Pharmaceutical Compositions,” all of which are hereby expressly incorporated by reference in their entirety.

### BACKGROUND OF THE INVENTION

Until recently, medical literature has recognized four types of insomnia, including sleep onset insomnia (e.g., trouble falling asleep at bedtime), sleep maintenance insomnia (e.g., disturbed sleep during the night), early morning awakening, and transient insomnia (e.g., new environment, first night in hotel syndrome). However, according to the National Sleep Foundation's 2005 “Sleep in America” poll, about 20% of total respondents and about 50% of respondents reporting insomnia symptoms complained of waking up too early and having difficulty returning to sleep at least a few nights a week (results available on the worldwide web at sleepfoundation.org). This type of insomnia includes “middle-of-the-night” insomnia, “late night” insomnia, “prolonged awakening after sleep onset” insomnia, “sleep maintenance” insomnia, and insomnia that follows after “middle-of-the-night” awakening, each of which has a component of interrupted sleep.

More particularly, patients with “middle-of-the-night” (MOTN) insomnia generally do not have problems initially falling asleep, but wake up prior to their intended wake time (during their normal sleep time), usually with about 3 to 4 hours of sleep time remaining. These patients require a treatment intervention that would reduce their wake time during their sleep time after awakening without leaving residual sedative effects in the morning. Unfortunately, currently available hypnotic medications are unsuitable for treating MOTN insomnia because they are slow to induce sleep (e.g., zaleplon) and/or require administration prior to about 7 to 9 hours in bed to avoid residual sleepiness in the morning (e.g., available dosage forms of zolpidem, eszopiclone, and zopiclone). Also, administration of most presently available hypnotics is prophylactic, which can result in unnecessary medication and overmedication of persons who require treatment for their MOTN insomnia a few nights a week. Other patients have recurring problems falling asleep and require a fast-acting sleep aid that can be taken prophylactically before going to bed.

Clearly, there remains a need for appropriate treatments for persons with MOTN insomnia and for those who need prophylactic treatment. The present invention fulfills this and other needs.

### BRIEF SUMMARY OF THE INVENTION

In a first embodiment, the present invention provides a pharmaceutical composition for buccal delivery of a therapeutic agent, the composition consisting essentially of (i) an effective amount of a weakly basic therapeutic agent; and (ii) a carbonate and bicarbonate buffer system in an amount sufficient to raise the pH of saliva to at least 8.5 irrespective of starting pH, and wherein the carbonate forms a coating on the bicarbonate wherein the amount of carbonate coating is at least 30% (w/w) of the total buffer amount.

In a second embodiment, the present invention provides a method of treating insomnia in a subject, the method comprising the step of administering to the subject the pharmaceutical composition described above, wherein the administering is on an as-needed basis.

In a third embodiment, the present invention provides a method of treating MOTN insomnia in a subject, the method comprising the step of administering to the subject a pharmaceutical composition described above, wherein the administering is on an as-needed basis, wherein the composition provides delivery of a therapeutic agent across the subject's oral mucosa.

### BRIEF DESCRIPTION OF THE DRAWINGS

NOT APPLICABLE

### DETAILED DESCRIPTION OF THE INVENTION

### I. General

The present invention provides compositions and methods for treating insomnia, using therapeutically effective low doses of zolpidem or a salt thereof by delivering zolpidem across the oral mucosa. The compositions of the present invention can be used to treat insomnia prophylactically, or for MOTN insomnia. The present invention is based, in part, upon the surprising discovery that low doses of zolpidem, when formulated for delivery across the oral mucosa, can induce rapid onset of sleep without residual sedative effects upon awakening 2-4 hours later. Advantages of taking a low dose amount of zolpidem less than 5 mg or 1.30×10⁻⁵ moles) to counteract MOTN insomnia include rapid action to induce sleep, treatment on an as-needed basis to avoid excessive and unnecessary medication, and no or minimal residual sedative effects upon awakening. The present invention further provides, in part, upon the surprising discovery that a binary buffer system using sodium bicarbonate and sodium carbonate wherein the sodium carbonate is at least 30% (w/w) of the buffer system and where the sodium carbonate coats the sodium bicarbonate, leads to improved stability of the compositions of the present invention.

While there are various types of dosage forms, solid dosage forms is for oral administration are perhaps among the most preferred by patients, and among the most prevalently used. Many of the dosage forms are medicaments formulated as tablets or capsules, which are swallowed. However, swallowed formulations have several disadvantages, including drug losses during hepatic first pass metabolism, during enzymatic degradation within the gastrointestinal tract, and during absorption to non-targeted tissues. These drug losses not only increase the variability in drug response, but also often require that the medicament be given in greater initial doses. Still further, as the drug has to pass through the gastrointestinal system in order to enter the blood stream, the time to reach a therapeutic effect may be quite long, typically around forty-five minutes or longer.

Drug delivery via the mucous membranes of the oral cavity has certain advantages, due to the properties of the oral mucosa itself. For example, the mucous membranes of the oral cavity are highly vascularized and well supplied with lymphatic drainage sites. In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

Accordingly, in certain aspects, the present invention provides pharmaceutical compositions containing low doses of zolpidem (e.g., dissolving tablets, lozenges, etc.) along with a binary buffer system with improved stability properties. Without being bound to any particular theory, the buffer system can promote the in situ conversion of a hydrophilic (i.e., charged) form of zolpidem (e.g., zolpidem hemitartrate) into its lipophilic free-base neutral) form, which penetrates the lipid membranes in the oral mucosa more readily than the salt form. The binary buffer system of the present invention achieves the improved stability by coating the bicarbonate with the carbonate such that the carbonate element of the binary buffer system is present in an amount of at least 30% (w/w) of the binary buffer system. As a result, both non-elderly and elderly patients can benefit from taking a substantially lower dose of zolpidem (e.g., about 3.5 mg for non-elderly; about 1.75 mg for elderly) as compared to the lowest currently approved dose of 5 mg, thereby rapidly inducing sleep without residual sedative effects upon awakening.

It is also desirable to reduce variability in drug delivery. Surprisingly, this can be achieved by utilizing a binary buffer system capable of achieving and sustaining a final pH in the oral cavity, independent of the initial pH. Accordingly, compositions for delivering zolpidem or a salt thereof across the oral mucosa having a buffer system providing increased stability and that produces a final pH, independent of the initial pH, and which sustains that final pH for a given period of time, are particularly desirable, and are provided herein.

### II. Definitions

As used herein, the following terms have the meanings ascribed to them unless specified otherwise.

As used herein, the term “therapeutic agent” refers to a drug that is administered to treat a condition, disease or illness. In the present invention, the therapeutic agent is used to treat insomnia either prophylactically or for MOTN insomnia. Therapeutic agents useful in the present invention include, but are not limited to, a hypnotic agent and a sedative. Suitable hypnotic agents for use in the present invention include, without limitation, an imidazopyridine compound such as zolpidem or alpidem; a dihydropyrrolopyrazine compound such as zopeclon; a pyrazolopyrimidine compound such as zaleplon or indiplon; pharmaceutically acceptable salts thereof; and combinations thereof. In a particularly preferred embodiment, the hypnotic agent is zolpidem, in all suitable forms. The therapeutic agents useful in the present invention can optionally be weakly basic. One of skill in the art will appreciate that other therapeutic agents are useful in the present invention.

As used herein, the term “hypnotic agent” refers to a drug that induces sleep, used in the treatment of insomnia. Hypnotic agents useful in the present invention include, but are not limited to, barbiturates, henzodiazepines, imidazopyridines, dihydropyrrolopyrazines, pyrazolopyrimidines, zolpidem, zaleplon, zopiclone, eszopiclone, chloral hydrate, chlormethiazole or the antihistamines doxylamine, promethazine, and diphenhydramine. One of skill in the art will appreciate that other hypnotic agents are useful in the present invention.

As used herein, the term “sleep disorder” refers to a disruptive pattern of sleep arising from many causes including, without limitation, dysfunctional sleep mechanisms, abnormalities in physiological functions during sleep, abnormalities of the biological clock, and sleep disturbances that are induced by factors extrinsic to the sleep process. In particular, the term encompasses disorders associated with difficulties in staying asleep and/or falling asleep such as insomnia (e.g., transient, short-term, and chronic), delayed sleep phase syndrome, hypnotic-dependent sleep disorder, and stimulant-dependent sleep disorder; disorders associated with difficulties in staying awake such as sleep apnea, narcolepsy, restless leg syndrome, obstructive sleep apnea, central sleep apnea, idiopathic hypersomnia, respiratory muscle weakness-associated sleep disorder; disorders associated with difficulties in adhering to a regular sleep schedule such as sleep state misperception, shift work sleep disorder, chronic time zone change syndrome, and irregular sleep-wake syndrome; disorders associated with abnormal behaviors such as sleep terror disorder (i.e., parasomnia) and sleepwalking (i.e., somnambulism); and other disorders such as sleep bruxism, fibromyalgia, and nightmares.

As used herein, the term “insomnia” refers to a sleep disorder characterized by symptoms including, without limitation, difficulty in falling asleep, difficulty in staying asleep, intermittent wakefulness, and/or waking up too early. The term also encompasses daytime symptoms such as sleepiness, anxiety, impaired concentration, impaired memory, and irritability. Types of insomnia suitable for treatment with the compositions of the present invention include, without limitation, transient, short-term, and chronic insomnia. The term “transient insomnia” refers to insomnia lasting for a few nights. The term “short-term insomnia” refers to insomnia lasting for about two to about four weeks. The term “chronic insomnia” refers to insomnia lasting for at least one month.

As used herein, the term “prolonged awakening after sleep onset insomnia” refers to the condition wherein a subject, after falling asleep, awakens and has difficulty returning to sleep, regardless of the number of hours of time in bed remaining. “Prolonged awakening after sleep onset insomnia” includes middle-of-the-night insomnia, late night insomnia, and insomnia after early night awakening.

As used herein, the term “middle-of-the-night insomnia” or “MOTN insomnia” refers to the condition wherein a subject, after falling asleep, awakens and has difficulty returning to sleep. Typically, the subject has about 5 hours of sleep time or time in bed remaining, although in some subjects only 4 hours, 3 hours, or 2 hours of sleep time may remain. One of skill in the art will appreciate that the term middle-of-the-night refers to a middle portion of the subjects sleep time in any sleep period, rather than a specific time of a time zone, day or night. For example, a shift worker who would normally sleep from Sam until 3 pm or 4 pm can still exhibit MOTN insomnia, when their sleep time is interrupted during normal daylight hours. MOTN insomnia can be transient, short-term, or chronic.

As used herein, the term “time in bed” refers to the amount of time a subject spends in a recumbent position lying down in bed or reclining in a chair) intending to sleep.

As used herein, the term “sleep time” refers to the time that a subject spends sleeping. Sleep time can be continuous or discontinuous.

As used herein, the term “sleep efficiency” refers to the total sleep time a subject receives during their time in bed. Sleep efficiency is measured by the following equation:

100*(total sleep time(TST)/total time in bed).

As used herein, the term “residual sedative effects” refers to a patient's subjective feeling of sedation upon awakening. Additionally, the term is meant to refer to a patient population as found in, for example, a clinical trial, rather than a single patient example. Residual sedative effects also can be evaluated using one or more of any of a number of tests exploring psychomotor performance, attention, information processing, and memory used by those of skill in the art including, for example, a Sleep Latency Test (SLT), a Visual Analog Test (VAT), a Digit Symbol Substitution Test (DSST), a Symbol Copying Test (SCT), a Critical Flicker Fusion threshold test (CFF), a Simple Reaction time test (visual or auditory; SRT), a Choice Reaction Time test (CRT), a Word Learning Test (WLT), a Critical Tracking Test (CTT), a Divided Attention Test (DAT), a digit or letter cancellation test, sleep staging through polysomnographic (PSG) measurements, Continuous Performance Task test (CPT), Multiple Sleep Latency Test (MSLT), a Rapid Visual Information Processing test (RVIP), a mental calculation test, a body sway test, a driving performance test, and others. Guidelines for a Sleep Latency Test are published in Sleep (1986) 9:519-24. The above-listed tests are described, for example, in Walsh, et al., (2000) Clin Neuropharm 23:17-21; Verster, et al., (2002) J. Psychopharm 22:576-583; Patat, et al, (2001) Human Psychopharm 16:369-392; and Hindmarch, et al., (2001) Human Psychopharm 16:159-167. As a result, an amount that substantially avoids or does not produce residual sedative effects is an amount that allows a subject, upon awakening following sleep time, to test acceptably in at least one of the above tests, preferably at least two or three of the above tests, and most preferably in at least four of the above tests.

Alternatively, an amount that substantially avoids or does not produce residual sedative effects can be objectively measured by determining the plasma or serum levels of zolpidem at an appropriate time point. In particular, residual sedative effects will be essentially extinguished when a subject's plasma levels of zolpidem fall below about 20 ng/ml. Again, this objective test refers to an average zolpidem plasma or serum concentration in a patient population. Because some variability between patients is expected, a number of patients may respond as having residual sedative effects even at low plasma or serum concentrations of zolpidem.

As used herein, the term “therapeutically effective amount” or “effective amount” refers to the amount of zolpidem that is capable of achieving a therapeutic effect in a subject in need thereof. For example, an effective amount of zolpidem can be the amount that is capable of preventing or relieving one or more symptoms associated with MOTN insomnia. It is important to note that a plasma concentration time curve for any given drug is illustrative of four, very often overlapping, kinetic events that decide the fate of the drug inside the body after the drug is administered. The four events are absorption, distribution, metabolism, and excretion. The absorption phase dominates in the beginning, while the distribution phase dominates at peak concentration time, and metabolism and excretion phases dominate the remaining disappearing stages of the drug. The sedative-hypnotic activity profile of zolpidem can be predicted from its plasma concentration time curve (Greenblatt et al., Clin. Pharmacol. Therap. 64:553 (1998)). In general, plasma concentrations between about 25 ng/ml and about 50 ng/ml, which are sufficient for inducing sleep, occur during the absorption phase of the drug, but this is not necessarily the peak concentration. Once the zolpidem is absorbed and distributed, the plasma concentrations will fall off with time. When the latter phase of drug distribution, metabolism, and excretion results in concentrations of zolpidem below about 20 ng/ml, the residual sedative effects of the drug will be essentially extinguished. This level will depend, to some extent, on the patient's age, hepatic efficiency, and initial dose. Generally, for the compositions and methods described herein, the sedative-hypnotic activity does not persist once the plasma levels have dropped below about 20 ng/ml, due to concurrence of continuous depiction of drug in the body and fulfillment of sleep requirement of the sleep-wake cycle of the body.

As used herein, the term “bioavailability” refers to the rate and/or extent to which a drug is absorbed or becomes available to the treatment site in the body. The MOTN efficacy zolpidem can also be improved by improving the bioavailability or the absorption of zolpidem, e.g., at rate of about 0.1 ng/ml per minute.

As used herein, the term “dissolves” or “dissolution” refers to the conversion of a portion of the pharmaceutical composition to a solution or slurry form. The amount of the pharmaceutical composition that dissolves over a period of time will vary depending on the components of the dosage form (e.g., the form of zolpidem used as well as the excipients used). Some pharmaceutical compositions will completely dissolve in a patient's mouth over a time period of about 15 minutes or less. Still other pharmaceutical compositions will completely dissolve in the mouth over a time period of about 6 minutes or less. Generally, at least about 25% by weight of the pharmaceutical composition will dissolve within about 5 minutes of administration. Suitable methods known in the art for determining the dissolution profile of a pharmaceutical composition include, e.g., United States Pharmacopeia (USP) dissolution tests such as USP <711> Apparatus 1 or USP <711> Apparatus 2.

As used herein, the term “disintegrates” or “disintegration” refers to the breakdown of, for example, a tablet or lozenge, into small pieces accompanied by complete dissolution of a substantial portion of the pharmaceutical composition to a liquid form. More particularly, disintegration of a pharmaceutical composition refers to less than about 25% by weight of the pharmaceutical composition remaining in the mouth following an appropriate time period, e.g., about 5 minutes after administration. Suitable methods known in the art for determining the disintegration profile of a pharmaceutical composition include, e.g., the USP disintegration test.

As used herein, the phrase “substantially complete conversion of zolpidem from its ionized to its un-ionized form” refers to greater than about 50% conversion of zolpidem from its ionized form into its un-ionized form. For example, a buffer system may favor at least about 50%, 55%, 60%, 65%, 70%, 75%, 80%, 85%, 90%, 95%, or 99% conversion of zolpidem from its ionized form into its un-ionized form. In some embodiments, the conversion occurs within about 10 minutes following administration.

As used herein, the term “variability” refers to inter-subject variability in terms of the percent of relative standard deviation (RSD) for the maximum plasma concentration (Cₘₐₓ) and the time to reach the maximum plasma concentration (Tₘₐₓ). Notably, the preferred compositions of the present invention have an RSD for Cₘₐₓ of about 33% versus about 45% for commercial oral tablets such as Ambien® tablets. Further, the compositions of the present invention have an RSD for Tₘₐₓ of about 50% or less versus about 100% for commercial oral tablets such as Ambien® tablets.

As used herein, the term “plasma concentration” refers to the concentration of therapeutic agent in the liquid component of the subject's blood.

As used herein, the term “subject” or “patient” refers to humans.

As used herein, the term “treating” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, the term “administering” refers to administration of the compositions of the present invention to the mucous membranes of the oral cavity (i.e., oral mucosa). Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. Preferably, the compositions of the present invention are administered to the sublingual mucosa, buccal mucosa, or a combination thereof.

### III. Description of the Embodiments

In some embodiments, the present invention provides a pharmaceutical composition for buccal delivery of a therapeutic agent, the composition essentially of (i) an effective amount of a weakly basic therapeutic agent; and (ii) a carbonate and bicarbonate buffer system in an amount sufficient to raise the pH of saliva to at least 8.5 irrespective of starting pH, and wherein the carbonate forms a coating on the bicarbonate wherein the amount of carbonate coating is at least 30% (w/w) of the total buffer amount.

In another embodiment, the present invention provides a pharmaceutical composition wherein the weakly basic therapeutic agent is a hypnotic agent selected from the group consisting of an imidazopyridine, a dihydropyrrolopyrazine, a pyrazolopyrimidine, and a pharmaceutically acceptable salt thereof.

In other embodiments, the present invention provides a pharmaceutical composition wherein the weakly basic therapeutic agent is zolpidem hemitartrate. In still other embodiments, the zolpidem hemitartrate is present in an amount of less than 5 mg. In yet other embodiments, the zolpidem hemitartrate is present in an amount less than 1.30×10⁻⁵ moles. In another embodiment, the zolpidem hemitartrate is present in an amount sufficient to produce a plasma concentration between about 25 ng/ml and about 50 ng/ml within 20 minutes of administration, when evaluated in an appropriate patient population.

In a further embodiment, the present invention provides a pharmaceutical composition wherein the buffer system produces a pH of at least 8.5 in a patient's saliva.

In some embodiments, the present invention provides a pharmaceutical composition wherein the carbonate coating is from 40% to 48% (w/w) of the total buffer amount.

In another embodiment, the present invention provides a pharmaceutical composition wherein the buffer system is present in particles having an average diameter of from 60 to 90 microns, in other embodiments, the buffer system is present in particles having an average diameter of from 70 to 80 microns.

In a further embodiment, the present invention provides a pharmaceutical composition wherein the buffer system comprises sodium carbonate and sodium bicarbonate.

In some embodiments, the present invention provides a pharmaceutical composition wherein the composition is a quick-dissolving lozenge or tablet. In another embodiment, the composition provides complete buccal dissolution in about 6 minutes or less following administration.

In other embodiments, the present invention provides a method of treating insomnia in a subject, the method comprising die step of administering to the subject the pharmaceutical composition described above, wherein the administering is on an as-needed basis.

In another embodiment, the present invention provides a method of treating MOTN insomnia in a subject, the method comprising the step of administering to the subject a pharmaceutical composition described above, wherein the administering is on an as-needed basis, wherein the composition provides delivery of a therapeutic agent across the subject's oral mucosa. In some embodiments, the subject is a subject who awakens from sleep and desires to resume sleep for less than 5 hours, and wherein the composition produces sleep within 30 minutes of dosing and the dose is such that it does not produce residual sedative effects when the subject is awakened at a time 4 hours after dosing. In a further embodiment, the subject takes the composition prophylactically before initial onset of sleep.

### IV. Compositions

The hypnotic agents of the present invention are preferably selected from an imidazopyridine compound such as zolpidem or alpidem; a dihydropyrrolopyrazine compound such as zopeclon; a pyrazolopyrimidine compound such as zaleplon or indiplon; pharmaceutically acceptable salts thereof; and combinations thereof. More preferably, the hypnotic agent is zolpidem, in all suitable forms.

In general, the hypnotic agents of the present invention are basic compounds having an ionized form and an un-ionized form. In certain instances, the hypnotic agent is initially present at least partly in an ionized form. In certain other instances, the hypnotic agent is initially present in an un-ionized form. As described in more detail below, the buffer system of the compositions described herein helps to convert substantially all of the hypnotic agent from its ionized form to its un-ionized form. Alternatively, the buffer system helps ensure that the hypnotic agent, initially in an un-ionized form, remains in an un-ionized form.

Conversion of the ionized form to the un-ionized forms for the hypnotic agent is related to pH according to the formula: pH=pKa+Log₁₀ (un-ionized concentration/ionized concentration). When the pH is the same as the pKa, equimolar concentrations of the un-ionized form and ionized form exist. For basic compounds such as the hypnotic agents described herein, when the pH is one unit higher than the pKa, the ratio of the un-ionized form to the ionized form is 91:9. Similarly, when the pH is two units higher than the pKa, the ratio of un-ionized form to the ionized form is 100:1. As noted above, the un-ionized form is lipophilic and, therefore, more capable of passing through mucous membranes such as the oral mucosa than the ionized form, which is lipophobic in nature. Accordingly, increasing the pH of the saliva favors conversion of the ionized form into the un-ionized form for basic compounds such as the hypnotic agents described herein, and the final pH can be determined by making use of the above formula.

The hypnotic agents of the present invention are selected from the class of compounds in the imidazopyridine, dihydropyrrolopyrazine, or pyrazolopyrimidine family and are useful in the treatment of conditions such as sleep disorders. Illustrative examples of suitable imidazopyridine compounds for use in the present invention are zolpidem, alpidem, pharmaceutically acceptable salts thereof, analogs thereof, and derivatives thereof. These imidazopyridine compounds each have an imidazopyridine group, as shown below:

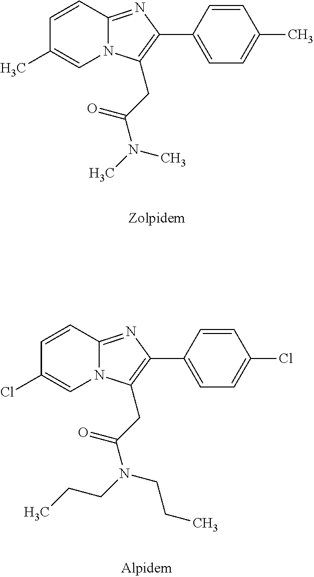

For the imidazopyridine compounds, the nitrogen in the imidazole portion of the bicyclic ring of the structure controls the extent of ionization and the degree of lipophilicity in any given medium. Typically the nitrogen in the imidazole portion imparts a pKa of from about 6.8 to about 7.5 to the molecule. Therefore, using the above formula, it can be demonstrated that about 90% conversion to an un-ionized form can be achieved for these compounds at a pH of from about 7.8 to about 8.5.

Illustrative examples of suitable dihydropyrrolopyrazine compounds for use in the present invention are zopeclon, pharmaceutically acceptable salts thereof, analogs thereof, and derivatives thereof. These dihydropyrrolopyrazines each have a dihydropyrrolopyrazine group, as shown below:

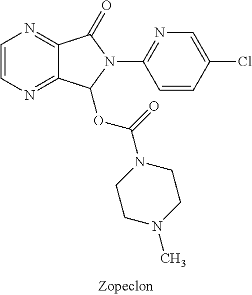

Illustrative examples of suitable pyrazolopyrimidine compounds for use in the present invention are zaleplon, indiplon, pharmaceutically acceptable salts thereof, analogs thereof, and derivatives thereof. These pyrazolopyrimidines each have a pyrazolopyrimidine group, as shown below:

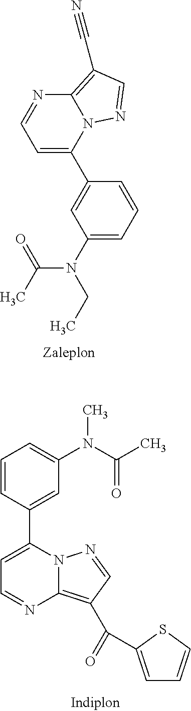

For the pyrazolopyrimidine compounds, the nitrogen in the pyrimidine group controls the extent of ionization and the degree of lipophilicity in any given medium. Typically, the nitrogen in the pyrimidine group imparts a pKa of from about 8 to about 9 to the molecule. Therefore, using the above formula, it can be demonstrated that about 90% conversion to an un-ionized form can be achieved for these compounds at a pH of from about 9 to about 10.

In general, the hypnotic agents of the present invention act as benzodiazepine receptor agonists. Preferably, the hypnotic agents selectively bind to the benzodiazepine receptor. Without being bound to any particular theory, the therapeutic activity of the hypnotic agents of the present invention in treating sleep disorders is attributed to an enhancement of the inhibitory action of gamma-aminobutyric acid (GABA) in the central nervous system. Alternatively, other therapeutic agents useful in the present invention include, but are not limited to, triptan drugs such as sumatriptan.

Typically, the compositions of the present invention will contain zolpidem or a salt thereof in an amount of about 0.5 mg, about 0.8 mg, about 1.0 mg, about 1.5 mg, about 1.75 mg, about 2.0 mg, about 2.5 mg, about 3.0 mg, about 3.5 mg, about 3.75 mg, about 4.0 mg, about 4.5 mg, or about 4.75 mg per administration. However, the amount of zolpidem can be any dose amount less than about 5 mg, alternatively from about 1.5 to about 2.5 mg, or alternatively from about 3.0 to about 3.75 mg. One skilled in the art will appreciate that the amount of zolpidem can be expressed as the number of moles of zolpidem present in the composition. For example, 5 mg of zolpidem hemitartrate is equivalent to about 1.30×10⁻⁵ moles of zolpidem. As such, in some embodiments, the composition will contain an amount of zolpidem hemitartrate that provides less than about 1.30×10⁻⁵ moles of zolpidem.

Any form of zolpidem is suitable for use in the compositions described herein, e.g., a salt form of zolpidem, a free base form of zolpidem, a polymorph of zolpidem, or a mixture thereof. For example, pharmaceutically acceptable salts of zolpidem can include, without limitation, tartrate, hemitartrate, succinate, dihydrochloride, salicylate, hemisuccinate, citrate, maleate, hydrochloride, carbamate, sulfate, nitrate, and benzoate salt forms, as well as combinations thereof. In some embodiments, the zolpidem is in the form of a salt, e.g., zolpidem hemitartrate. In other embodiments, the zolpidem is in the form of a polymorph, e.g., commercially available from Plantex Ltd. (Netanya, Israel).

The compositions of the present invention may take the form of solid, semi-solid, lyophilized powder, or liquid dosage forms, such as, for example, tablets (e.g., chewable, slow-dissolving, quick-dissolving, etc.), pills, capsules, lozenges, gums, powders, solutions, suspensions, emulsions, aerosols, foams, creams, gels, lotions, or the like. Preferably, the compositions of the present invention are formulated as a tablet or a lozenge, in particular quick-dissolving tablets or lozenges, such as those described in U.S. Patent Publication No. 200500226925.

As used herein, the term “unit dosage” or “dosage form” refers to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients such as carriers. Methods for preparing such dosage forms are known or will be apparent to those skilled in the art. For example, in some embodiments, a chewing gum dosage form of the present invention can be prepared according to the procedures set forth in U.S. Pat. No. 4,405,647. In other embodiments, a liquid spray or a solution, tincture, tablet, lozenge, or candy dosage form of the present invention can be prepared according to the procedures set forth, for example, in Remington: The Science and Practice of Pharmacy, 20^{th} Ed., Lippincott, Williams & Wilkins (2003); Pharmaceutical Dosage Forms, Volume 1: Tablets, 2^{nd} Ed., Marcel Dekker, Inc., New York, N.Y. (1989); and similar publications. The dosage form to be administered will, in any event, contain a quantity of the therapeutic agent in a therapeutically effective amount for relief of the condition being treated when administered in accordance with the teachings of the present invention.

The terms “carrier” or “excipient” refer to a typically inert substance used as a diluent or vehicle for a drug such as a therapeutic agent. The term also encompasses a typically inert substance that imparts cohesive qualities to the composition. Suitable carriers for use in the compositions of the present invention include, without limitation, a binder, a gum base, and combinations thereof. Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyoxyethylene polymers, polyacrylic acid (e.g., Carbopol), calcium silicate, calcium phosphate, dicalcium phosphate, calcium sulfate, kaolin, sodium chloride, polyethylene glycol, propylene glycol, and combinations thereof. These binders can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant, dusting and wet-granulation preparation with a suitable lubricating agent (see, e.g., Remington: The Science and Practice Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the binder, and preferably from about 50% to about 80%. However, one skilled in the art will appreciate that the compositions of the present invention can be made without any binders, e.g., to produce a highly friable dosage form.

Non-limiting examples of gum bases include materials selected from among the many water-insoluble and saliva-insoluble gum base materials known in the art. For example, in some instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000). Typically, the gum base comprises from about 25% to about 75% by weight of these polymers, and preferably from about 30% to about 60%.

The compositions of the present invention can additionally include lubricating agents; wetting agents; emulsifying agents; solubilizing agents; suspending agents; preserving agents such as methyl-, ethyl-, and propyl-hydroxy-benzoates, butylated hydroxytoluene, and butylated hydroxyanisole; sweetening agents; flavoring agents; coloring agents; and disintegrating agents such as crospovidone as well as croscarmellose sodium and other cross-linked cellulose polymers.

Lubricating agents can be used to prevent adhesion of the dosage form to the surface of the dies and punches, and to reduce inter-particle friction. Lubricating agents may also facilitate ejection of the dosage form from the die cavity and improve the rate of granulation flow during processing. Examples of suitable lubricating agents include, without limitation, magnesium stearate, calcium stearate, zinc stearate, stearic acid, sodium stearyl fumarate, simethicone, silicon dioxide, talc, hydrogenated vegetable oil, polyethylene glycol, mineral oil, and combinations thereof. The compositions of the present invention can comprise from about 0% to about 10% by weight of the lubricating agent, and preferably from about 1% to about 5%.

Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Of the foregoing, sorbitol, mannitol, and xylitol, either alone or in combination, are preferred sweetening agents. The compositions of the present invention can comprise from about 0% to about 80% by weight of the sweetening agent, preferably from about 5% to about 75%, and more preferably from about 25% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein, Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

When the dosage form is a chewing gum, the composition can comprise zolpidem or a pharmaceutically acceptable salt thereof (“therapeutic agent”), a carrier or excipient such as a gum base, a pH-adjusting agent or buffer system, and optionally a protecting agent. The chewing gum composition may further comprise lubricating agents, wetting agents, emulsifying agents, solubilizing agents suspending agents, preserving agents, sweetening agents, flavoring agents, and coloring agents. Typically, the chewing gum composition comprises less than about 5 mg (e.g., from about 0.5 mg to about 4.75 from about 1.5 mg to about 2.5 mg, from about 3.0 mg to about 3.75 mg, etc.) of zolpidem or a salt thereof. One skilled in the art understands that the foregoing amounts will vary depending upon the particular source of zolpidem utilized, the amount of zolpidem desired in the final formulation, as well as on the particular release rate of zolpidem desired. In certain instances, the buffer system of the chewing gum composition provides a final salivary pH in excess of at least about 7.8, preferably at least about 8.5, and more preferably at least about 9 (e.g., about 9-11). The chewing gum composition typically comprises from about 20% to about 95% by weight of the gum base, more typically from about 30% to about 85%, and most typically from about 50% to about 70% of the gum base.

The chewing gum composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of from about 1 to about 10. Without being bound to any particular theory, the protecting agent reduces the adhesion between the therapeutic agent and the gum base so that the therapeutic agent may be more easily released from the gum base. In this way, the therapeutic agent may be delivered across the mucous membranes of the oral cavity within about 5 to about 20 minutes of chewing, preferably within about 10 minutes of chewing. A variety of different protecting agents may be used. Examples of suitable protecting agents include, without limitation, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, sodium stearyl fumarate, mineral oil, poloxamer, polyethylene gycol, sodium benzoate, sodium chloride, sodium lauryl sulfate, stearic acid, cab-o-sil, talc, zinc stearate, and combinations thereof.

The gum base may additionally include plasticizers such as softeners or emulsifiers. Such plasticizers may, for example, help reduce the viscosity of the gum base to a desirable consistency and improve its overall texture and bite. Plasticizers may also facilitate the release of the therapeutic agent upon mastication. Non-limiting examples of plasticizers include lecithin, mono- and diglycerides, stearic acid, sodium stearate, potassium stearate, glycerol triacetate, glycerol monostearate, glycerin, and combinations thereof. The gum base typically comprises from about 0% to about 20% by weight of the plasticizer, and more typically from about 5% to about 15%.

The gum base may further comprise waxes such as beeswax and microcrystalline wax, fats or oils such as soybean and cottonseed oil, and combinations thereof. Typically, gum base comprises from about 0% to about 25% by weight of these waxes and oils, and more typically comprises from about 15% to about 20%.

In addition, the gum base may further comprise one or more elastomeric solvents such as rosins and resins. Non-limiting examples of such solvents include methyl, glycerol, and pentaerythritol esters of rosins, modified rosins such as hydrogenated, dimerized or polymerized rosins, or combinations thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin such as polymers of alpha-pinene or beta-pinene, terpene resins including polyterpene, and combinations thereof). Typically, the gum base comprises from about 0% to about 75% by weight of the elastomeric solvent, and more typically less than about 10%.

The gum base may further comprise a filler material to enhance the chewability of the final chewing gum composition. Fillers that are substantially non-reactive with other components of the final chewing gum formulation are preferable. Examples of suitable fillers include, without limitation, calcium carbonate, magnesium silicate (i.e., talc), dicalcium phosphate, metallic mineral salts (e.g., alumina, aluminum hydroxide, and aluminum silicates), and combinations thereof. Typically, the gum base comprises from about 0% to about 30% by weight of the filler, and more typically from about 10% to about 20%.

One skilled in the art will appreciate that the gum base need not be prepared from its individual components. For example, the gum base can be purchased with the desired ingredients contained therein, and can be modified to include additional agents. Several manufacturers produce gum bases suitable for use with the described chewing gum compositions. Examples of such gum bases include, without limitation, Pharmagum™ M, S, or C(SPI Pharma Group; New Castle, Del.). In general, Pharmagum™ comprises a mixture of gum base, sweetening agent, plasticizer, and sugar.

In certain instances, the chewing gum composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the gum base surrounds, at least in part, a centerfill. The centerfill comprises at least one therapeutic agent, and may be a liquid or semi-liquid material. The centerfill material can be a synthetic polymer, a semi-synthetic polymer, low-fat, or fat-free and contain one or more sweetening agents, flavoring agents, coloring agents, and/or scenting agents. Preferably, the centerfill includes a buffer system as described herein. Methods for preparing a centerfill chewing gum are described, for example, in U.S. Pat. No. 3,806,290.

The chewing gum compositions can have any desired shape, size, and texture. For example, the chewing gum can have the shape of a stick, tab, gumball, and the like. Similarly, the chewing gum can be any desirable color. For example, the chewing gum can be any shade of red, blue, green, orange, yellow, violet, indigo, and mixtures thereof, and can be color coded to indicate the type and dosage of the therapeutic agent therein. The chewing gum can be individually wrapped or grouped together in pieces for packaging by methods well known in the art.

When the dosage form is a tablet such as a dissolving tablet or chewable tablet, the composition can comprise zolpidem or a pharmaceutically acceptable salt thereof, a carrier or excipient such as a binder, and a pH-adjusting agent or buffer system. The tablet composition may further comprise protecting agents, lubricating agents, wetting agents, emulsifying agents, solubilizing agents; suspending agents, preserving agents, sweetening agents, flavoring agents, coloring agents, and disintegrating agents. Typically, the tablet compositions of the present invention comprise less than about 5 mg (e.g., from about 0.5 mg to about 4.75 mg, from about 1.5 mg to about 2.5 mg, from about 3.0 mg to about 3.75 mg, etc.) of zolpidem or a salt thereof. One skilled in the art understands that the foregoing amounts will vary depending upon the particular source of zolpidem utilized, the amount of zolpidem desired in the final formulation, as well as on the particular release rate of zolpidem desired. In certain instances, the buffer system of the tablet compositions provide a final salivary pH in excess of at least about 7.8, preferably at least about 8.5, and more preferably at least about 9 (e.g., about 9-11).

In certain embodiments, the tablet is a dissolving tablet such as a slow-dissolving or quick-dissolving tablet that is dissolved by a subject's saliva, without the need for chewing. For example, a dissolving tablet placed on the subject's tongue can be used for buccal delivery of the therapeutic agent. Alternatively, a dissolving tablet placed underneath the subject's tongue can be used for sublingual delivery of the therapeutic agent. This type of dosage form may be particularly desirable for pediatric and geriatric patients, since small children and aged individuals often have difficulty chewing certain items. Typically, the dissolving tablet is formulated to dissolve within about 1 to about 15 minutes, preferably within about 2 to about 10 minutes, e.g., within about 2, 3, 4, 5, 6, 7, 8, 9, or 10 minutes, following administration. One skilled in the art will understand that quick-dissolving tablets dissolve faster than slow-dissolving tablets, which are typically dissolved gradually rather than rapidly by a subject's saliva. In a preferred embodiment, the slow-dissolving or quick-dissolving tablet delivers the therapeutic agent across the sublingual mucosa.

In certain other embodiments, the tablet is a chewable tablet that is chewed by a subject and formulated to dissolve either rapidly or gradually. For example, a chewable tablet placed on the subject's tongue can be used for buccal delivery of the therapeutic agent. During chewing, the chewable tablet can be moved around within the mouth and can sometimes be parked between the gums and the cheeks or underneath the tongue. As a result, at least a portion of the therapeutic agent contained within a chewable tablet may also be delivered sublingually (i.e., across the sublingual mucosa). Typically, the chewable tablet is formulated to dissolve within about 1 to about 15 minutes, preferably within about 2 to about 10 minutes, e.g., within about 2, 3, 4, 5, 6, 7, 8, 9, or 10 minutes, following administration.

As described above, the dissolving and chewable tablets of the present invention are typically formulated to dissolve within about 1 to about 15 minutes following administration. However, while these time frames are amenable to maximum exposure of the therapeutic agent to the oral mucosa (e.g., to the sublingual and/or buccal mucosa), they are not always amenable to user compliance e.g., users may swallow too frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the tablet size (e.g., from about 700-800 mg to about 200-300 mg or about 100-350 mg) without reducing the concentration or amount per unit dose of the buffer system or the therapeutic agent. In addition, subtle changes to the tablet formulation such as, for example, replacing one flavoring agent for another (e.g., chocolate for spearmint) or replacing one binder or sweetening agent for another (e.g., lactose for mannitol or sorbitol) may be used to reduce salivation.

The carrier or excipient present in the tablets of the present invention is typically a binder that is useful in keeping the tablet in a semi-solid state, and may be a solid or a liquid, and may for example be a high-melting point fat or waxy material. Materials suitable as binders are discussed in detail above and may be used alone or in combination in the tablet compositions of the present invention. In addition, binders such as mannitol, sorbitol, lactose, sucrose, and inositol can impart properties to the tablet that permit or enhance its disintegration in the mouth.

The tablet composition may also comprise one or more elastomeric solvents such as rosins and resins. Non-limiting examples of such solvents are discussed in detail above and may be used alone or in combination in the tablet compositions of the present invention. In addition, the tablet composition may further comprise waxes such as beeswax and microcrystalline wax, fats or oils such as soybean and cottonseed oil, and combinations thereof. Moreover, the tablet composition may additionally include plasticizers such as softeners or emulsifiers. Such plasticizers may, for example, help reduce the viscosity of the salivary solution of the dissolved tablet to a desirable consistency and improve its overall texture and bite and help facilitate the release of the therapeutic agent. Non-limiting examples of such plasticizers are discussed in detail above and may be used alone or in combination in the tablet compositions of the present invention.

In certain instances, the tablet composition includes a therapeutic agent centerfill, e.g., as described above. In certain other instances, the tablet composition of the present invention is multilayered. In this way, the dissolving or chewable tablet can be designed to provide more than one therapeutic agent. For example, with a bi-layered tablet, the first layer can contain zolpidem or a salt thereof and the second layer can contain the same or different hypnotic agent or a non-hypnotic agent. Typically, the first layer comprises the dissolving or chewable portion of the tablet, and the second (i.e., subsequent) layer is coated by the first layer. This type of formulation may be particularly suitable when immediate release of zolpidem, followed by gastrointestinal absorption of a second therapeutic agent, is desirable. Gastrointestinal absorption of the second therapeutic agent may be desirable, for example, in order to mitigate co-morbid symptoms or to sustain the therapeutic benefit of zolpidem in the dissolving or the chewable portion of the tablet. Alternatively, the second layer is present as a layer lateral to the first layer. The second layer typically comprises at least one therapeutic agent, and can also comprise one or more sweetening agents, flavoring agents, coloring agents, and scenting agents as described above. In some instances, the second layer further includes a buffer system as described herein.

In still other instances, the combination of zolpidem or a salt thereof with other hypnotic agents and/or non-hypnotic agents need not take the form of a multilayered tablet, but instead comprises a single homogenous tablet layer. This type of formulation may also be used in the case where gastrointestinal absorption of at least one therapeutic agent is desirable. In this case, the relative extent of ionization of the two or more therapeutic agents determines how they are to be absorbed. For example, those therapeutic agents that are un-ionized are absorbed through the oral mucosa, while the ionized agents are swallowed for gastrointestinal absorption.

The tablet compositions can have any desired shape, size, and texture. For example, the tablet can have the shape of a stick, tab, pellet, sphere, and the like. Similarly, the tablet can be any desirable color. For example, the tablet can be any shade of red, blue, green, orange, yellow, violet, indigo, and mixtures thereof, and can be color coded to indicate the type and dosage of the therapeutic agent therein. The tablets can be individually wrapped or grouped together in pieces for packaging by methods well known in the art.

When the dosage form is a lozenge or candy, the composition can comprise zolpidem or a pharmaceutically acceptable salt thereof, a carrier or excipient such as a binder, and a pH-adjusting agent or buffer system. The lozenge or candy composition may further comprise protecting agents, lubricating agents, wetting agents, emulsifying agents, solubilizing agents; suspending agents, preserving agents, sweetening agents, flavoring agents, coloring agents, and disintegrating agents. A general discussion of lozenges and candies is provided, e.g., in Pharmaceutical Dosage Forms, Volume 1: Tablets, 2^{nd} Ed., Marcel Dekker, Inc., New York, N.Y., pages 75-418 (1989). Typically, the lozenge compositions of the present invention comprise less than about 5 mg (e.g., from about 0.5 mg to about 4.75 mg, from about 1.5 mg to about 2.5 mg, from about 3.0 mg to about 3.75 mg, etc.) of zolpidem or a salt thereof. One skilled in the art understands that the foregoing amounts will vary depending upon the particular source of zolpidem utilized, the amount of zolpidem desired in the final formulation, as well as on the particular release rate of zolpidem desired. In certain instances, the buffer system of the lozenge compositions provides a final salivary pH in excess of at least about 7.8, preferably at least about 8.5, and more preferably at least about 9 (e.g., about 9-11).

In certain embodiments, the lozenge or candy is dissolved by a subject's saliva, without the need for chewing. For example, a lozenge placed on the subject's tongue can be used for buccal delivery of the therapeutic agent. Alternatively, a lozenge placed underneath the subject's tongue can be used for sublingual delivery of the therapeutic agent. This type of dosage form may be particularly desirable for pediatric and geriatric patients, since small children and aged individuals often have difficulty chewing certain items. Typically, the lozenge is formulated to dissolve within about 1 to about 15 minutes, preferably within about 2 to about 10 minutes, e.g., within about 2, 3, 4, 5, 6, 7, 8, 9, or 10 minutes, following administration. In a preferred embodiment, the lozenge or candy delivers the therapeutic agent across the sublingual mucosa.

As described above, the lozenges of the present invention are typically formulated to dissolve within about 1 to about 15 minutes following administration. However, while these time frames are amenable to maximum exposure of the therapeutic agent to the oral mucosa (e.g., to the sublingual and/or buccal mucosa), they are not always amenable to user compliance (e.g., users may swallow too frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the lozenge size (e.g., from about 700-800 mg to about 200-300 mg or about 100-350 mg) without reducing the concentration or amount per unit dose of the buffer system or the therapeutic agent. In addition, subtle changes to the lozenge formulation such as, for example, replacing one flavoring agent for another (e.g., chocolate for spearmint) or replacing one binder or sweetening agent for another (e.g., lactose for mannitol or sorbitol) may be used to reduce salivation.

The carrier or excipient present in the lozenges of the present invention is typically a binder that is useful in keeping the lozenge in a semi-solid state, and may be a solid or a liquid, and may for example be a high-melting point fat or waxy material. Materials suitable as binders are discussed in detail above and may be used alone or in combination in the lozenge compositions of the present invention. In addition, binders such as mannitol, sorbitol, lactose, sucrose, and inositol can impart properties to the lozenge that permit or enhance its disintegration in the mouth.

The lozenge composition may also comprise one or more elastomeric solvents such as rosins and resins. Non-limiting examples of such solvents are discussed in detail above and may be used alone or in combination in the lozenge compositions of the present invention. In addition, the lozenge composition may further comprise waxes such as beeswax and microcrystalline wax, fats, or oils such as soybean and cottonseed oil, and combinations thereof. Moreover, the lozenge composition may additionally include plasticizers such as softeners or emulsifiers. Such plasticizers may, for example, help reduce the viscosity of the salivary solution of the dissolved lozenge to a desirable consistency and improve its overall texture and bite and help facilitate the release of the therapeutic agent. Non-limiting examples of such plasticizers are discussed in detail above and may be used alone or in combination in the lozenge compositions of the present invention.

In other embodiments, the lozenge composition includes a therapeutic agent centerfill, is multilayered, or comprises a single homogenous lozenge layer, as described in detail above.

The lozenge compositions can have any desired shape, size, and texture. For example, the lozenge can have the shape of a stick, tab, pellet, sphere, and the like. Similarly, the lozenge can be any desirable color. For example, the lozenge can be any shade of red, blue, green, orange, yellow, violet, indigo, and mixtures thereof, and can be color coded to indicate the type and dosage of the therapeutic agent therein. The lozenges can be individually wrapped or grouped together in pieces for packaging by methods well known in the art.

In a preferred embodiment, the average particle size of the drug in the compositions described herein is about 20 microns, as compared to a typical average drug particle size of from about 75 to about 100 microns. In another preferred embodiment, the average particle size of the drug in the compositions described herein is less than or equal to the average particle size of the carrier ingredients (e.g., gum base, binders, etc).

Typically, the pharmaceutical compositions are suitable for buccal or sublingual administration of zolpidem in the low doses provided herein. Compositions suitable for buccal or sublingual administration of zolpidem are those that provide absorption in the buccal cavity of at least about 10%, 20%, or 25% of the dosage of zolpidem in the composition. This amount is generally at least twice the amount of buccal absorption that could be expected for a tablet designed to be swallowed for absorption of the active agent in the gut. Additionally, the time to Cₘₐₓ is reduced for such compositions relative to tablets or capsules designed to deliver zolpidem in the gut. The compositions suitable for buccal or sublingual administration of zolpidem in low doses, as noted above, are sufficient to reduce the time to Cₘₐₓ, enhancing the early effect of zolpidem and increase plasma levels of zolpidem, generally two-fold or more during the first 20 minutes after administration, relative to tablets or capsules designed for delivery in the gut (e.g., to be swallowed immediately upon ingestion).

Typically, the compositions that are suitable for the treatment of insomnia following buccal or sublingual administration have a unique and discriminatory dissolution profile. Such a dissolution method relies on modified USP method II dissolution procedure and where the pH of the dissolution medium is 6.8, which approximates the pH of the saliva. The method is considered to be a modification as the volume of the medium is reduced to 500 ml from 900 ml and the paddle speed for dissolution is reduced to 15 rpm from a typical speed of 50 or more rpm. This method is sufficiently sensitive to discriminate a 2 to 3 minute dissolution tablet from a tablet that would normally take 5 minutes or more to dissolve in the mouth. Typically, a tablet that would dissolve in the mouth in 3 minutes or less would dissolve more rapidly under experimental conditions of modified USP method II than a tablet that takes 5 or more minutes to dissolve in the mouth (see, Tables 1-2 below)

TABLE 1
Exploratory dissolution profiles of 3 and 5 minute dissolution of
zolpidem lozenges using the modified USP dissolution method II.

| | “3 minute” dissolution | | “5 minute” dissolution | |
|---|---|---|---|---|
| Lozenge | prototype | | prototype | |
| Time (min) | Dissolution | RSD* | Dissolution | RSD* |
| 5 | 28.60% | 5% | 8.70% | 12.00% |
| 10 | 58.40% | 10% | 20.00% | 11.30% |
| 20 | 79.00% | 20% | 38.30% | 11.40% |

(500 ml of pH 6.8 phosphate buffer at a 37° C. and paddle speed of 15 rpm).
*Relative standard deviation

TABLE 2
Illustrative dissolution profiles of 1, 3.5, and 10 mg “3 minute”
zolpidem lozenges using the modified USP dissolution method II.
(500 ml of pH 6.8 phosphate buffer at a 37° C. and paddle
speed of 15 rpm).

| | Lozenge | | | | | |
|---|---|---|---|---|---|---|
| | 1 mg | | 3.5 mg | | 10 mg | |
| | “3 minute” | | “3 minute” | | “3 minute” | |
| | dissolution | | dissolution | | dissolution | |
| | prototype | | prototype | | prototype | |
| | Dis- | | Dis- | | Dis- | |
| Time (min) | solution | RSD | solution | RSD | solution | RSD |
| 5 | 28.70% | 11.60% | 42.40% | 11.14% | 28.60% | 19.90% |
| 10 | 46.90% | 9.30% | 70.20% | 6.53% | 58.40% | 10.40% |
| 15 | 60.40% | 6.70% | 81.00% | 7.23% | | |
| 20 | 70.50% | 5.20% | 84.30% | 7.14% | 79.00% | 5.10% |

In some embodiments, the compositions of the present invention provide complete buccal and/or sublingual dissolution in about 2 minutes or less following administration. The quick-dissolving tablets of the present invention usually provide complete buccal and/or sublingual dissolution in less than about 0.5 minutes, alternatively in less than about minute, alternatively in less than about 1.5 minutes, alternatively in less than about 2 minutes, alternatively in less than about 2.5 minutes, alternatively in less than about 3 minutes, alternatively in less than about 4 minutes, alternatively in less than about 5 minutes, or alternatively in less than about 6 minutes.

Generally, the compositions described herein may comprise a binary buffer system comprising a bicarbonate core surrounded by a layer of carbonate. Alternatively, the coating material may not completely surround the core. As a further alternative, the buffered soda may comprise a material in which sodium carbonate and sodium bicarbonate are co-located in a single particle. The carbonate/bicarbonate buffer system of the present invention comprises at least one proton donating (acidic) component and at least one proton accepting (basic) component. The amounts of the carbonate and bicarbonate components of the buffer system are selected such that the buffering capacity is greatest (the buffer system has a pK value) at a pH of from about 7.2-11.0, usually at a pH of about, for example, 7.2, 7.6, 7.8, 8.0, 8.3, 8.5, 8.8, 9.0, 9.4, 9.5, 9.6, 9.7, or 9.8.

In preferred embodiments, the binary buffer system raises the pH of saliva to a pH greater than about 7.2, 7.6, 7.8, 8.0, 8.3, 8.5, or 8.8, irrespective of the starting pH of saliva. In other embodiments, the binary buffer system raises the pH of saliva to a pH greater than about 9.0, 9.4, 9.5, 9.6, 9.7, or 9.8 (e.g., about 9-11), irrespective of the starting pH of saliva.

Preferably, the buffer system comprises a carbonate and a bicarbonate component. For example, the carbonate salt can be selected from the group consisting of sodium carbonate, potassium carbonate, calcium carbonate, ammonium carbonate, and magnesium carbonate. The bicarbonate salt can be selected from the group consisting of sodium bicarbonate, potassium bicarbonate, calcium bicarbonate, ammonium bicarbonate, and magnesium bicarbonate. In a preferred embodiment, the binary buffer system comprises sodium carbonate and sodium bicarbonate. In another preferred embodiment, the sodium bicarbonate is desiccant-coated sodium bicarbonate. The cations of the carbonate and the bicarbonate components can be the same or different.

The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes. This typically involves a sensory and safety trial and error type of procedure of adding various amounts of each buffer system component and then measuring the final pH over time. In this way, selection of an appropriate weight ratio for each buffer system component can be determined.

In some embodiments, the binary buffer system used in the compositions described above comprises a carbonate salt such as sodium carbonate and a bicarbonate salt such as sodium bicarbonate, wherein the carbonate salt and the bicarbonate salt are in a carbonate:bicarbonate ratio of from about 1:1.0 to about 1:1.6 by weight, or alternatively from about 1:1.2 to about 1:1.4 by weight.

In another embodiment, the composition of the present invention is stabilized using a binary buffer system where the bicarbonate component is coated with the carbonate component. Such a binary buffer system can be prepared by several methods known to one of skill in the art. Some buffer systems useful in the present invention are commercially available, such as from SPI Pharma of New Castle, Del. Buffer systems useful in the present invention can be prepared according to the procedure of U.S. Pat. No. 3,105,792. In some embodiments, the amount of carbonate is at least 30% (w/w) of the binary buffer system. In some other embodiments, the amount of carbonate is at least 40% (w/w) of the binary buffer system. In another embodiment, the amount of carbonate is at least 40.5%, 41.0%, 42.5%, 43.0%, 43.5%, 44.0%, 44.5% or 45.0%, or greater, of the binary buffer system. One of skill in the art will appreciate that other binary buffer system compositions are useful in the present invention.

In other embodiments, the bicarbonate can be used by itself to promote selective absorption of zolpidem.

In still other embodiments, the pharmaceutical compositions comprise a carrier comprising at least one binder and at least one disintegrating agent in such relative proportion to provide a buccal or sublingual dissolution time of about 5 minutes or less, preferably about 2 minutes or less, following administration. Preferably, the ratio of the binder to the disintegrating agent is from about 0.1 to about 10.0, more preferably from about 0.1 to about 1.0, and most preferably from about 0.26 to about 0.79. However, one skilled in the art will appreciate that the compositions of the present invention can be made without any binders, to produce a highly friable dosage form.

In a preferred embodiment, the zolpidem is delivered across an oral mucosa selected from the group consisting of the sublingual mucosa, the buccal mucosa, and a combination thereof. In a particularly preferred embodiment, the composition is administered sublingually so that the zolpidem is delivered across the sublingual mucosa.

In preferred embodiments of the present invention, the zolpidem is formulated in a binary buffer system comprising sodium carbonate and sodium bicarbonate. Such compositions are preferably formulated in the form of a lozenge, candy, or dissolving tablet (e.g., slow-dissolving tablet or quick-dissolving tablet) for sublingual administration. As a result, upon sublingual administration, zolpidem is delivered across the sublingual mucosa. In another preferred embodiment, the combined weight percent of the sodium carbonate and sodium bicarbonate buffer system is greater than or equal to the weight percent of zolpidem.

In some embodiments, the composition comprises from about 0.4, 0.45, or 0.5 to about 1.5, 1.6, 1.7, or 1.8 weight percent zolpidem. In a preferred embodiment, the composition comprises about 0.47, 0.8, or 1.7 weight percent zolpidem; about 8.0 weight percent sodium carbonate; and about 11.0 weight percent desiccant-coated sodium bicarbonate. Such compositions are preferably in the form of a lozenge or candy with a mass of from about 100 to about 300 mg, e.g., about 100, 110, 120, 130, 140, 150, 160, 170, 180, 190, 200, 210, 220, 230, 240, 250, 260, 270, 280, 290, and 300 mg. The lozenges or tablets dissolve in a subject's mouth at a very rapid rate, e.g., within about 2-3 minutes following administration.

In certain other instances, the composition is preferably in the form of a dissolving tablet such as a slow-dissolving tablet or a quick-dissolving tablet of from about 100 to about 300 mg, e.g., about 100, 110, 120, 130, 140, 150, 160, 170, 180, 190, 200, 210, 220, 230, 240, 250, 260, 270, 280, 290, and 300 mg. The quick-dissolving tablets dissolve in a subject's mouth at a rapid rate, e.g., within about 5 minutes following administration, and the slow-dissolving tablets dissolve in a subject's mouth at a slower rate, e.g., within about 10 minutes following administration.

### V. Methods

In carrying out the methods of the present invention for treating insomnia, the appropriate effective dosage to be administered to a subject can be evaluated in an appropriate patient population that has been selected based on factors such as age, weight, the number of hours of time in bed remaining, and/or the ability of a subject to metabolize zolpidem. Accordingly, effective amounts of zolpidem for delivery across the oral mucosa may be different for selected patient populations. For example, the effective amount of zolpidem in an elderly patient population (i.e., subjects 65 years of age and older) is usually from about 1.5 mg to about 2.5 mg of zolpidem, alternatively about 1.75 mg, alternatively about 2.0 mg, or alternatively about 2.5 mg. Similarly, the effective amount of zolpidem in a population of subjects with a diminished capacity to metabolize zolpidem can be from about 1.5 mg to about 2.5 mg of zolpidem, alternatively about 1.75 mg, alternatively about 2.0 mg, or alternatively about 2.5 mg. The effective amount of zolpidem in a non-elderly patient population (i.e., subjects younger than 65 years of age) is usually from about 3.0 mg to about 3.75 mg zolpidem, alternatively about 125 mg, alternatively about 3.5 mg, or alternatively about 3.75 mg. The effective amount of zolpidem in subjects who have awakened but still have about 4 or 5 hours of time in bed remaining can be from about 2 mg to about 5 mg of zolpidem. A lower amount of zolpidem (e.g., from about 0.5 mg to about 2.5 mg, alternatively about 0.5 mg, alternatively about 1.0 mg, alternatively about 1.5 mg, alternatively about 2.0 mg, or alternatively about 2.5 mg) can be administered to subjects who have awakened but still have about 2 to 4 hours of time in bed remaining. Likewise, subjects with a diminished capacity to metabolize zolpidem (i.e., subjects 65 years of age and older) can be administered a portion of a dose that would be administered to a subject with a normal capacity to metabolize zolpidem, for example, a half-tablet dose. One of skill in the art will appreciate that there can be some variability in the dose provided to some individuals. For example, hepatically-impaired individuals may use a very low dose such as that typically provided for an elderly patient.

Any method known in the art can be used to determine the plasma concentration of zolpidem in a subject. As a non-limiting example, the plasma from a blood sample collected from the subject can be assayed for zolpidem levels using high pressure liquid chromatography (HPLC) followed by tandem mass spectrometry (MS) or fluorescence detection. Chromatographic methods for measuring plasma levels of zolpidem are described in, for example, Ascalone et al., J. Chromatogr., 581:237-250 (1992); Tracqui et al., J. Chromatogr., 616:95-103 (1993); Durol et al., J. Anal. Toxicol., 215:388-392 (1997); Ptacek et al., J. Chromatogr. B Biomed. Sci. Appl., 694:409-413 (1997) and Ring et al., J. Pharm. Biomed. Anal., 22:495-504 (2000).

Typically, an effective amount; of zolpidem is administered to a subject with insomnia on an as needed basis, i.e., pro re nata. That is, the individual had previously fallen asleep, and the sleep time has been interrupted with at least about 2, 3, 4, or 5 hours of time in bed remaining.

Alternatively, an effective amount of zolpidem is administered to a subject on an as needed basis prophylactically before the initial onset of sleep. For example, the individual can have a recurrent problem falling asleep. Or, the individual can have a need to ensure falling asleep quickly.

Typically, the methods are carried out by administering a composition of the present invention as described above. Compositions of particular interest for treating insomnia contain less than about 5 mg of zolpidem or a salt thereof. In certain embodiments, the zolpidem can be administered in a quick-dissolving tablet or lozenge. Efficient delivery of zolpidem can be achieved using a formulation with a binary or a ternary buffer system, for example with carbonate and bicarbonate components, as described above.

Administration of the compositions of the present invention is preferably carried out via any of the accepted modes of administration to the mucous membranes of the oral cavity. Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. These regions differ from each other with respect to their anatomy, drug permeability, and physiological response to drugs. Preferably, the compositions of the present invention are administered to the sublingual mucosa, buccal mucosa, or a combination thereof.

The oral mucosa, possessing a rich blood supply and suitable drug permeability, is an especially attractive route of administration for systemic drug delivery. Furthermore, delivery of a therapeutic agent across the oral mucosa bypasses hepatic first pass metabolism, avoids enzymatic degradation within the gastrointestinal tract, and provides a more suitable enzymatic flora for drug absorption. As used herein, the term “sublingual delivery” refers to the administration of a therapeutic agent across the mucous membranes lining the floor of the mouth and/or the ventral tongue. The term “buccal delivery” as used herein refers to the administration of a therapeutic agent across the mucous membranes lining the cheeks.

### VI. Examples

The following examples are offered to illustrate, but not to limit, claimed invention.

### Example 1

### Preparation of Zolpidem Lozenge Compositions

Low dose zolpidem lozenge compositions are prepared according to the formulations set forth in Table 3.

TABLE 3
Low dose zolpidem lozenge formulations.

| | Quantity (mg/lozenge) | | | |
|---|---|---|---|---|
| | Strength | | | |
| Component | 1.0 mg | 1.75 mg | 1.75 mg | 3.5 mg |
| Zolpidem hemitartrate | 1.0 | 1.75 | 1.75 | 3.5 |
| Pharmaburst ™ B2 | | | | |
| consisting of: | | | | |
| mannitol | 142 | 70 | 141.25 | 139.5 |
| sorbitol | | | | |
| crospovidone | | | | |
| silicon dioxide | | | | |
| Croscarmellose | 10 | 5 | 10 | 10 |
| sodium | | | | |
| Buffer system (sodium | 40 | 20 | 40 | 40 |
| carbonate + sodium | | | | |
| bicarbonate) | | | | |
| Natural and artificial | 6.5 | 3.25 | 6.5 | 6.5 |
| spearmint FONA# | | | | |
| 913.004 | | | | |
| Silicon dioxide | 5.5 | 2.75 | 5.5 | 5.5 |
| Sucralose | 1.5 | 0.75 | 1.5 | 1.5 |
| Magnesium stearate | 3.5 | 1.75 | 3.5 | 3.5 |
| Total lozenge weight | 210 | 105 | 210 | 210 |

The sodium carbonate/sodium bicarbonate buffer system can be obtained from SPI Pharma in New Castle, Del., prepared according to U.S. Pat. No. 3,105,792.

### Example 2

### Low Dose Zolpidem Tablet Composition

An immediate release peroral (PO) tablet containing a low dose of zolpidem can be prepared according to the formulation set forth in Table 4.

TABLE 4
Low dose zolpidem tablet formulation.

| Component | Quantity (mg) |
|---|---|
| Zolpidem Hemitartrate | 3.5 |
| Povidone K29/32 | 15.0 |
| Sodium Starch Glycolate (SSG) | 7.5 |
| Starch 1500 | 15.0 |
| Lactose Fast Flow | 82.0 |
| Prosolv SMCC 90 | 65.5 |
| Buffer system (sodium carbonate + | 40 |
| sodium bicarbonate) | |
| Magnesium Stearate | 1.5 |
| Total | 230 |

The sodium carbonate/sodium bicarbonate buffer system can be obtained from SPI Pharma in New Castle, Del., prepared according to U.S. Pat. No. 3,105,792.

### Manufacturing Process

Dispensing: Screen the zolpidem hemitartrate and excipients through screen #30. Dispense the required quantities of each ingredient.

Blending:
Transfer the zolpidem hemitartrate and Povidone K 29/32 to a V-Shell blender and blend for 2 min.
2. Add SSG and Starch 1500 to Step 1 and blend for another 2 ruin.
3. Add Lactose Fast Flow and Prosolv SMCC 90 to Step 2 and blend for another 10 min.
4. Mix an equal amount of the blend from Step 3 with magnesium stearate or sodium stearyl fumarate and transfer the mixture back to the V-Shell blender via screen #30. Blend for 3 min.

Compression: Compress the final blend from Step 4 on a rotary press to a target tablet weight of 210 mg.

### Example 3

### Zolpidem Tartrate Lozenge Compositions

Using the methods and procedures above, additional lozenge compositions were prepared according to the specifications set forth in Table 5. Table 5 provides 4 formulations made according to this invention, with varying amounts of the lubricant, sodium stearyl fumarate, and silicon dioxide. Formulation 145 has a lower amount of the lubricant, sodium stearyl fumarate, and is advantageous when using a simple embossing pattern on the tablet/compressed lozenge where the issue of the tablet/compressed lozenge sticking to the punches is not present.

If a complex pattern is used to identify the product, or if such a pattern is desirable for marketing purposes, sticking to the punches can be avoided by using large amounts of sodium stearyl fumarate. In addition, by the use of large amounts of silicon dioxide, sticking and moisture sensitivity are reduced. In these formulations, 10 mg of the total lozenge mass of 210 mg consists of silicon dioxide.

Large amounts of silicon dioxide can be incorporated in the form of fine particle material, such as Cab-O-Sil. Incorporation of large amounts of silicon dioxide as fine particles can be difficult in large scale manufacture. Mixing is facilitated by the use of a different physical form of this chemical. Syloid, due to its larger particle size, is easier to handle and incorporate. The porous nature of Syloid, specifically Syloid 244 FP grade, allows it to trap moisture, and is thus suitable for use in the present invention.

Syloid 244 FP consists of larger particles than Cab-O-Sil, thus, it is easier to blend into the tableting mixture, as in formula 136. However, some of the anti-adherent properties of fine particle silicon dioxide is lost if Syloid is used as the exclusive source of silicon dioxide. Anti-adherence is a desired feature, accordingly, in some instances, it is preferable to incorporate the silicon dioxide as a combination of each of the two physical forms, i.e. larger particle Syloid 244 FP and very fine particle Cab-O-Sil. This is exemplified in formula 165 in which 2 mg Cab-O-Sil and 8 mg Syloid are used per lozenge. Cab-O-Sil products useful in the present invention, include Cab-O-Sil M 5.

Buffered soda is the trade name given to the product comprising sodium carbonate and sodium bicarbonate where the sodium carbonate forms an outer coating around the sodium bicarbonate, and the product comprises 43% (w/w) sodium carbonate.

TABLE 5
Zolpidem Tartrate Lozenge Compositions

| | mg/tab | mg/tab | mg/tab | mg/tab |
|---|---|---|---|---|
| Batch No. | 145 | 150 | 136 | 165 |
| Zolpidem Tartrate | 3.50 | 3.50 | 3.50 | 3.50 |
| Buffered Soda | 40.00 | 40.00 | 40.00 | 40.00 |
| Pharmaburst B2 | 135.75 | 131.00 | 131.00 | 131.00 |
| Croscarmellose Na | 10.00 | 10.00 | 10.00 | 10.00 |
| Yellow Iron oxide | 1.00 | 1.00 | 1.00 | 1.00 |
| Sucralose | 1.50 | 1.50 | 1.50 | 1.50 |
| Spearmint Flavor | 3.00 | 3.00 | 3.00 | 3.00 |
| Silicon Dioxide (Cab-O-Sil) | 10.00 | 10.00 | — | 2.00 |
| Silicon Dioxide (Syloid 244 FP) | — | — | 10.00 | 8.00 |
| Sodium stearyl fumarate | 5.25 | 10.00 | 10.00 | 10.00 |
| Total Wt. of Lozenge | 210.00 | 210.00 | 210.00 | 210.00 |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition for buccal and/or sublingual delivery of a therapeutic agent, said composition comprising:
an effective amount of zolpidem; and
a carbonate and bicarbonate buffer system in an amount sufficient to raise the of saliva to at least 8.5, and wherein the carbonate forms a coating on the bicarbonate wherein the amount of carbonate coating is at least 30% (w/w) of the total buffer amount.

2. The pharmaceutical composition of claim 1, wherein the zolpidem is zolpidem hemitartrate.

3. The pharmaceutical composition of claim 2, wherein the zolpidem hemitartrate is present in an amount of less than 5 mg.

4. The pharmaceutical composition of claim 2, wherein the zolpidem hemitartrate is present in an amount less than 1.30×10⁻⁵ moles.

5. The pharmaceutical composition of claim 1, wherein the buffer system produces a pH of at least 8.5 in a patient's saliva.

6. The pharmaceutical composition of claim 1, wherein the carbonate coating is from 40% to 46% (w/w) of the total buffer amount.

7. The pharmaceutical composition of claim 1, wherein the buffer system is present in particles having an average diameter of from 60 to 90 microns.

8. The pharmaceutical composition of claim 1, wherein the buffer system comprises sodium carbonate and sodium bicarbonate.

9. The pharmaceutical composition of claim 1, wherein the composition is a quirk-dissolving lozenge or tablet.

10. A pharmaceutical composition for buccal and/or sublingual delivery of a therapeutic agent, said composition comprising:
an effective amount of zolpidem; and
a binary buffer system comprising carbonate and bicarbonate, wherein the carbonate and bicarbonate are co-located in a single particle, wherein the bicarbonate is coated with the carbonate, wherein the amount of carbonate coating is at least 30% (w/w) of the binary buffer system.

11. The pharmaceutical composition of claim 10, wherein the zolpidem is zolpidem hemitartrate.

12. The pharmaceutical composition of claim 11, wherein the zolpidem hemitartrate is present in an amount of less than 5 mg.

13. The pharmaceutical composition of claim 11, wherein the zolpidem hemitartrate is present in an amount less than 1.30×10⁻⁵ moles.

14. The pharmaceutical composition of claim 10, wherein the buffer system produces a pH of at least 8.5 in a patient's saliva.

15. The pharmaceutical composition of claim 10, wherein the carbonate coating is from 40% to 48% (w/w) of the total buffer amount.

16. The pharmaceutical composition of claim 10, wherein the buffer system is present in particles having an average diameter of from 60 to 90 microns.

17. The pharmaceutical composition of claim 10, wherein the buffer system comprises sodium carbonate and sodium bicarbonate.

18. The pharmaceutical composition of claim 10, wherein the composition is a quick-dissolving lozenge or tablet.
